(19) United States
(12) Patent Application Publication
(10) Pub. No.: US 2012/0277538 A1
(43) Pub. Date: Nov. 1, 2012

(54) BODY INSERTION INSTRUMENT

(75) Inventor: Yuta OKADA, Tokyo (JP)

(73) Assignee: OLYMPUS CORPORATION, Tokyo (JP)

(21) Appl. No.: 13/546,295

(22) Filed: Jul. 11, 2012

Related U.S. Application Data
(62) Division of application No. 12/576,665, filed on Oct. 9, 2009.
Application No. 12/576,665 is a continuation of application No. PCT/JP2008/057161, filed on Apr. 11, 2008.

(30) Foreign Application Priority Data
Apr. 12, 2007 (JP) 2007-105242

(51) Int. Cl. A61B 1/32

(52) U.S. Cl. 600/204

(57) ABSTRACT

A body insertion instrument has an inserting portion inserted into a body near a target organ as a target of surgery. The body insertion instrument further includes a space securing unit, disposed to the inserting portion, configured to secure a space, in which the target organ is operated on, one of between the target organ and an organ different from the target organ and between the target organ and one of an abdominal wall and a chest wall, and a drive unit configured to drive the space securing unit for acting force the one of between the target organ and the organ different from the target organ and between the target organ and the one of the abdominal wall and the chest wall.

### CROSS REFERENCE TO RELATED APPLICATIONS

This application is a divisional of U.S. patent application Ser. No. 12/576,665, filed on Oct. 9, 2009, which is a continuation application of International Application No. PCT/JP2008/057161, filed on Apr. 11, 2008, which was published under PCT Article 21(2) in Japanese, and is based upon and claims the benefit of priority from prior Japanese Patent Application No. 2007-105242, filed on Apr. 12, 2007, the entire contents of each of which are incorporated herein by reference.

### BACKGROUND OF THE INVENTION

1. Field of the Invention

The present invention relates to a body insertion instrument inserted into a body in surgery.

2. Description of the Related Art

In conventional heart surgery, a surgical instrument is permitted to access a chest cavity by dissecting in a chest bone (median sternotomy). In this case, a retractor is disposed in the opened portion of the chest and widens a portion between a chest bone and tissues so that a large opening is formed thereto. Then, a surgical instrument is disposed through the opening and the heart surgery is performed.

One of the most common forms of heart surgery is coronary artery bypass grafting (CABG). In CABG, when one or a plurality of coronary arteries is blocked, the blocked portions are bypassed by connecting a transplanted blood vessel (graft) to a coronary artery downstream of the blocked portions. A technique for connecting a graft to a coronary artery is known as anastomosis. For example, a chest artery dissected from a chest wall is used as the graft, and, in this case, an upstream end of the chest artery remains without being injured, and a downstream end of the chest artery is connected to the coronary artery. Further, an artery or a vein from any portion of a patient's body may be used as the graft. Further, an artificial blood vessel graft can be also used. In this case, an upstream end of the graft is connected to an artery such as the aorta, and a downstream end thereof is connected to a coronary artery. As described above, blocked portions of a plurality of coronary arteries at various positions on front, side, and back surfaces of a heart are bypassed using a plurality of grafts.

Conventionally, since CABG is performed by stopping a patient's heart, the patient's blood is circulated using an artificial heart-lung machine.

However, CABG may be performed while the heart is beating by a technique known as “off pump coronary artery bypass” (OPCAB), by which use of an artificial heart-lung machine can be avoided.

In OPCAB, a surface of a heart near to an anastomosis portion of a coronary artery is fixed using a special instrument called a stabilizer while the heart is beating. The anastomosis portion is kept so that it does not move as far as possible by partially fixing the anastomosis portion by the stabilizer while the graft is being connected to the coronary artery.

As disclosed in, for example, International Publication No. WO 01/054562, the stabilizer described above has a contact portion in contact with an organ and a flexible contact portion support portion for supporting the contact portion. The contact portion support portion is formed of joint members so that it can bend and deform, and slender cables such as wires extend passing through inside of the joint members. The contact portion support portion is inserted into the chest cavity while being bent and deformed by appropriately adjusting the tensions of the cables. Then, movement of the heart is stabilized by causing the contact portion to come into contact with a desired portion of the heart and to push or draw the heart.

In median sternotomy and thoracotomy, since a large opening is formed by widening a portion between a chest bone and tissues by a retractor, a surgeon can directly observe a state of the stabilizer.

Further, when an anastomosis portion is a portion located on a back surface side and the like of a heart which cannot be viewed from a front surface thereof in an ordinary state, a position of the heart is adjusted so that the anastomosis portion can be observed while drawing and holding the heart by a surgical instrument disclosed in, for example, U.S. Patent Application Publication No. 2005/0049463.

In contrast, recently, an endoscopic operation is performed also by CABG to carry out various treatments in a body cavity by forming a hole to a body cavity wall such as an abdominal wall and inserting an endoscope and a surgical instrument into the body cavity as a minimally invasive surgery which does not require significant dissection. In this case, an observation camera is inserted into a body cavity at a position thereof corresponding to a diseased portion shown in a CT image acquired before an operation is performed, and the operation is performed while observing an operating portion.

In the endoscopic operation, a retractor is used to secure a field of view of an observation camera by opening flat plates in a fan shape after they are inserted into a body cavity and eliminating under pressure an organ other than an organ to be operated on by the flat plates opened in the fan shape as disclosed in, example, Jpn. Pat. Appln. KOKAI Publication No. 6-154152.

### BRIEF SUMMARY OF THE INVENTION

According to an aspect of the present invention, there is provided a body insertion instrument comprising:

an inserting portion inserted into a body near a target organ as a target of surgery;

a space securing unit, disposed to the inserting portion, configured to secure a space, in which the target organ is operated on, one of between the target organ and an organ different from the target organ and between the target organ and one of an abdominal wall and a chest wall; and

a drive unit configured to drive the space securing unit for acting force the one of between the target organ and the organ different from the target organ and between the target organ and the one of the abdominal wall and the chest wall.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1A is a view showing a structure of a body insertion instrument according to a first embodiment of the present invention.

FIG. 1B is a view showing a state that a contact portion of the body insertion instrument is deformed in an arc-shape.

FIG. 1C is a view showing a state that a balloon of the body insertion instrument is inflated.

FIG. 2 is a view showing a configuration of an operation system using the body insertion instrument according to the first embodiment.

FIG. 3 is a block diagram showing an electric configuration of the operation system using the body insertion instrument according to the first embodiment.

FIG. 4A is a view showing a structure of a body insertion instrument according to a second embodiment of the present invention.

FIG. 4B is a front elevational view of a body insertion instrument according to the second embodiment.

FIG. 5 is a view showing a structure of a body insertion instrument according to a third embodiment of the present invention.

FIG. 6A is a view showing a structure of a body insertion instrument according to a first modification of the third embodiment.

FIG. 6B is a view showing a state that an opening/closing portion of the body insertion instrument according to the first modification is opened.

FIG. 7 is a view showing a structure of a body insertion instrument according to a second modification of the third embodiment.

FIG. 8 is a view showing a structure of a body insertion instrument according to a third modification of the third embodiment.

FIG. 9 is a view showing a structure of a body insertion instrument according to a fourth embodiment of the present invention.

### DETAILED DESCRIPTION OF THE INVENTION

The best modes for carrying out the present invention will be described below referring to drawings.

### First Embodiment

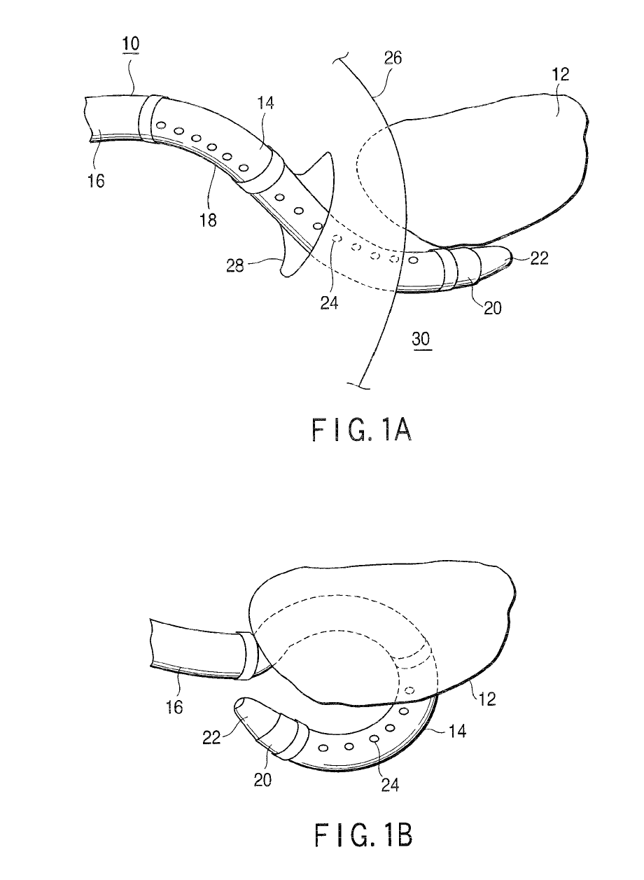

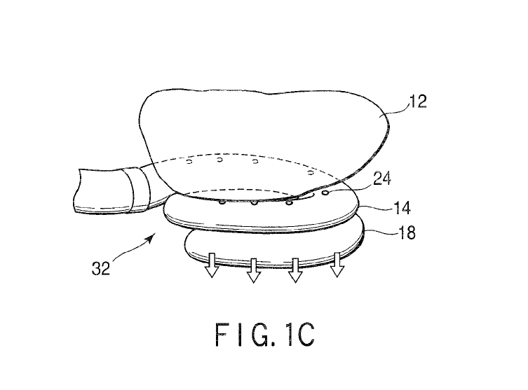

As shown in FIG. 1A, a body insertion instrument 10 according to a first embodiment of the present invention has a flexible contact portion 14, which is inserted into a body near a target organ 12 such as a heart and comes into contact with the target organ 12, a flexible contact portion support portion 16 for supporting the contact portion 14, and a balloon 18 disposed on a side opposite to a side where the contact portion 14 is in contact with the target organ 12.

The contact portion 14 and the contact portion support portion 16 are formed of, for example, joint members so that the contact portion 14 and the contact portion support portion 16 can be bent and deformed. Slender cables (not shown) such as wires extend through inside of the joint members. Further, a small camera 20 is assembled to an extreme end of the contact portion 14. Further, a transparent taper hood 22 is attached to the small camera 20 to secure a field of view of the camera 20 and to protect it. The transparent taper hood 22 improves insertability of the contact portion 14 into the body. Further, suction holes 24 are formed on a side of the contact portion 14 in contact with the target organ 12, and a suction air pressure transmission path (not shown) communicating with the suction holes 24 extends to the outside through the contact portion 14 and the contact portion support portion 16.

In contrast, the balloon 18 is attached to the contact portion 14 in a contracted state so that it does not disturb the body insertion instrument 10 when it is inserted into the body. The balloon 18 is connected to a supply air pressure transmission path (not shown), which extends to the outside through inside of the contact portion support portion 16, and can be inflated by supplying air from the supply air pressure transmission path into the balloon 18. Since the balloon 18 is disposed along a lengthwise direction of the contact portion 14, it is inflated in the same curved shape as that of the contact portion 14.

The body insertion instrument 10 is positioned by a body insertion instrument support arm (not shown). Thereafter, the body insertion instrument 10 is inserted into a chest cavity 30 through a hole formed on a body cavity wall (not shown) and a hole 28 formed on a diaphragm 26 by bending and deforming the contact portion support portion 16 by appropriately adjusting tensions of the cables while observing the body insertion instrument 10 by the small camera 20. As a result, the contact portion 14 is caused to come into contact with a desired portion of the target organ 12. Thereafter, the body insertion instrument 10 is further inserted while deforming the contact portion 14 in a doughnut shape by appropriately adjusting the tension of the cable.

Then, as shown in FIG. 1B, when the contact portion 14 is deformed in an arc-shape, and the respective suction holes 24 are placed in a desired state in which they face the target organ 12, suction force is applied to the target organ 12 through the suction air pressure transmission path to thereby draw the target organ 12 by the suction holes 24. Together with the above operation, air is supplied into the balloon 18 through the supply air pressure transmission path into the balloon 18 and inflates it. With this operation, expansion force of the balloon 18 shown by arrows in FIG. 1C acts as separation force to an adjacent organ (not shown), which is different from the target organ 12 and exists on the lower side (the opposite side of the side on which the contact portion 14 is in contact with the target organ 12) of the balloon 18 or to a body cavity wall (not shown) such as an abdominal wall, a chest wall. The target organ 12 is separated from the adjacent organ or the body cavity wall by the separation force, and an operation space 32 continuous to the chest cavity 30 is secured between the target organ 12 and the adjacent organ or the body cavity wall. Note that the amount of the expansion of the balloon 18 can be adjusted while observing the balloon by the small camera 20. Further, since the contact portion 14 draws the target organ 12 by the suction force of the suction holes 24 as well as is pushed to the target organ 12 by the separation force of the balloon 18, it can stabilize the motion of the target organ 12.

As described above, the body insertion instrument 10 according to the present embodiment can perform a function of a stabilizer for stabilizing the motion of the target organ 12 as well as also a function for securing the operation space 32.

Further, since the body insertion instrument 10 according to the embodiment can be configured to have a small volume when it is inserted into a body, it can be easily inserted thereinto.

Note that it is needless to say that the suction force of the suction holes 24 and the amount of expansion of the balloon 18 can be appropriately adjusted during an operation. Further, it is needless to say that when the operation is completed, the suction force is released as well as the balloon 18 is contracted and the body insertion instrument 10 is extracted from inside the body.

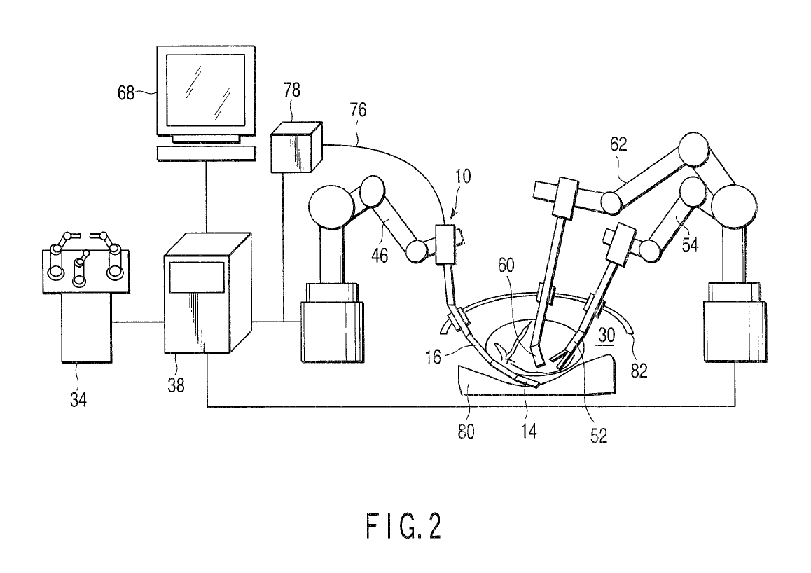

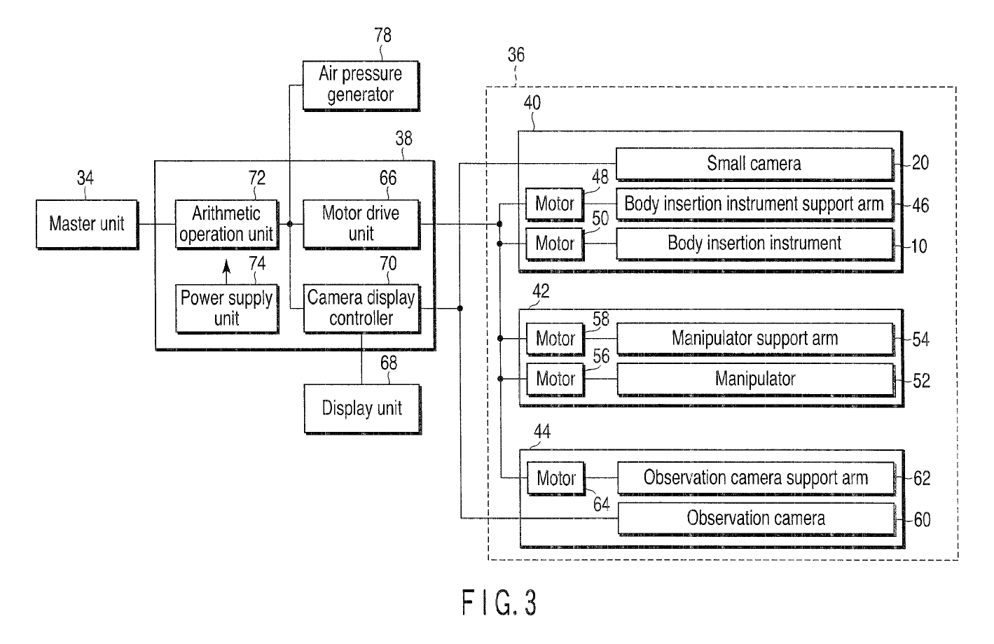

As shown in FIGS. 2 and 3, an operation system using the body insertion instrument 10 configured as described above has, for example, a master unit 34 as a remote operation unit and a controller 38 for controlling a slave main body 36 based on remote operation information from the master unit 34.

The slave main body 36 includes a body insertion instrument slave unit 40, a manipulator slave unit 42, and an observation camera unit 44.

The body insertion instrument slave unit 40 includes the body insertion instrument 10, the small camera 20, and the body insertion instrument support arm 46. Further, the body insertion instrument slave unit 40 includes a motor 48 for driving respective drive mechanism portions (not shown) in the body insertion instrument 10 and a motor 50 for driving respective drive mechanism portions (not shown) in the body insertion instrument support arm 46.

The manipulator slave unit 42 includes a manipulator 52 for operating on a portion in the body. Note that since the manipulator 52 is disclosed in, for example, U.S. Pat. No. 5,876,325 in detail, description thereof is omitted. The manipulator slave unit 42 further includes a manipulator support arm 54 for supporting the manipulator 52 and positioning it to an operating portion, a motor 56 for driving respective drive mechanism portions (not shown) in the manipulator 52, and a motor 58 for driving respective drive mechanism portions (not shown) in the manipulator support arm 54.

The observation camera unit 44 includes an observation camera 60 for observing a portion in the body, an observation camera support arm 62 for supporting the observation camera 60 and positioning it with respect to an operating portion, and a motor 64 for driving respective drive mechanism portions (not shown) in the observation camera support arm 62.

In contrast, the controller 38 includes a motor drive unit 66 for driving the respective motors 48, 50, 56, 58, 64 of the slave main body 36 and a camera display controller 70 for controlling operation of the observation camera 60 and an image display operation of a display unit 68 connected to the controller 38. Further, the controller 38 includes an arithmetic operation unit 72 for moving and operating the body insertion instrument 10, the manipulator 52, and the observation camera 60 by creating a signal for controlling the amount of movement and operation of the body insertion instrument 10, the manipulator 52, and the observation camera 60 based on remote manipulation information from the master unit 34 and supplying the signal to the motor drive unit 66 and the camera display controller 70. Further, the controller 38 includes a power supply unit 74 for supplying power to the respective units.

The arithmetic operation unit 72 creates a signal for controlling operation of an air pressure generator 78 for generating air pressure applied to an air pressure transmission path 76 based on the remote operation information from the master unit 34 and supplying the signal to the air pressure generator 78. As a result, a drawing operation of the suction holes 24 of the contact portion 14 of the body insertion instrument 10 and an expansion/contraction operation for expanding and contracting the balloon 18 are performed. Note that the air pressure transmission path 76 is branched to two types of paths in it, i.e., to the suction air pressure transmission path and the supply air pressure transmission path so that the air pressures of them can be independently controlled.

Accordingly, a surgeon inserts the body insertion instrument 10 into the chest cavity 30 by manipulating the master unit 34 while observing an image of an operating portion captured by the observation camera 60 and displayed on the display unit 68 and an image captured by the small camera 20 attached to the extreme end of the contact portion 14 of the body insertion instrument 10. Then, the surgeon can stabilize the motion of the target organ 12 by causing the contact portion 14 of the body insertion instrument 10 to come into contact with a desired portion of the target organ 12 as well as can secure the operation space 32 between the target organ 12 and an adjacent organ 80 or a body cavity wall. Thereafter, the surgeon can operate on a diseased portion of the target organ 12 by inserting the manipulator 52 through a costal bone 82 into the operation space 32 secured in a gap between the extreme end of the contact portion 14 (and the balloon 18), which is deformed in an arc-shape, of the body insertion instrument 10 and the contact portion support portion 16, by manipulating the master unit 34 like the body insertion instrument 10.

Further, according to the body insertion instrument 10, all the action points and the counteraction points exist in the body cavity because the expansion force of the balloon 18 acts on a side opposite to the side where the contact portion 14 is in contact with the target organ 12. Since force for expanding the balloon 18 is generated in the body insertion instrument 10, it is not applied to the outside (for example, to the body). Accordingly, since rigidity of the body insertion instrument 10 need not be increased, it can be composed of a soft member or can be made compact in its entirety.

### Second Embodiment

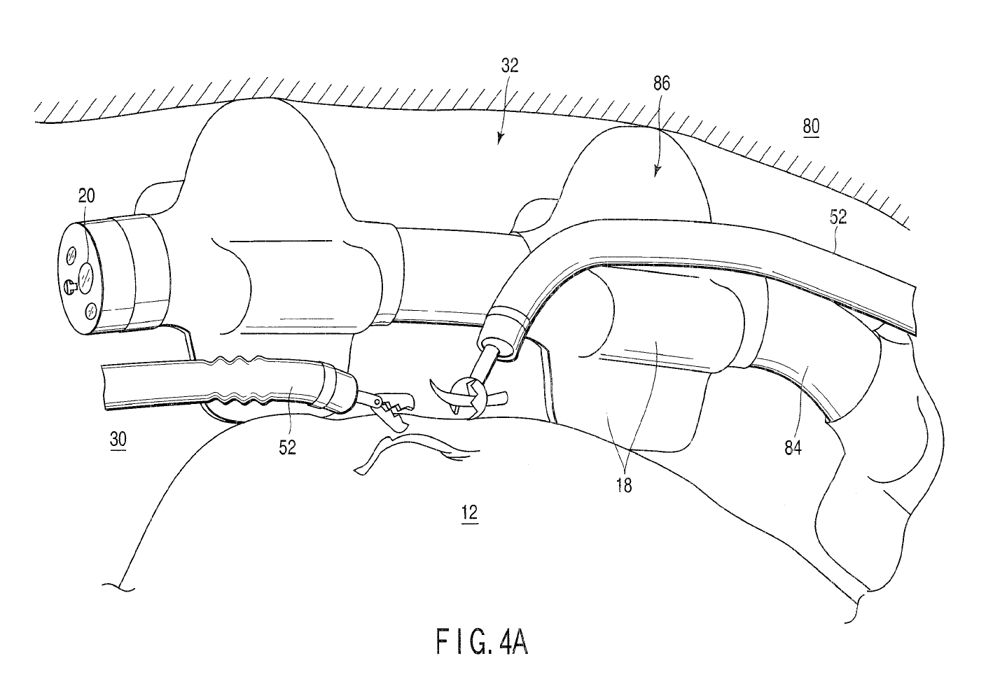

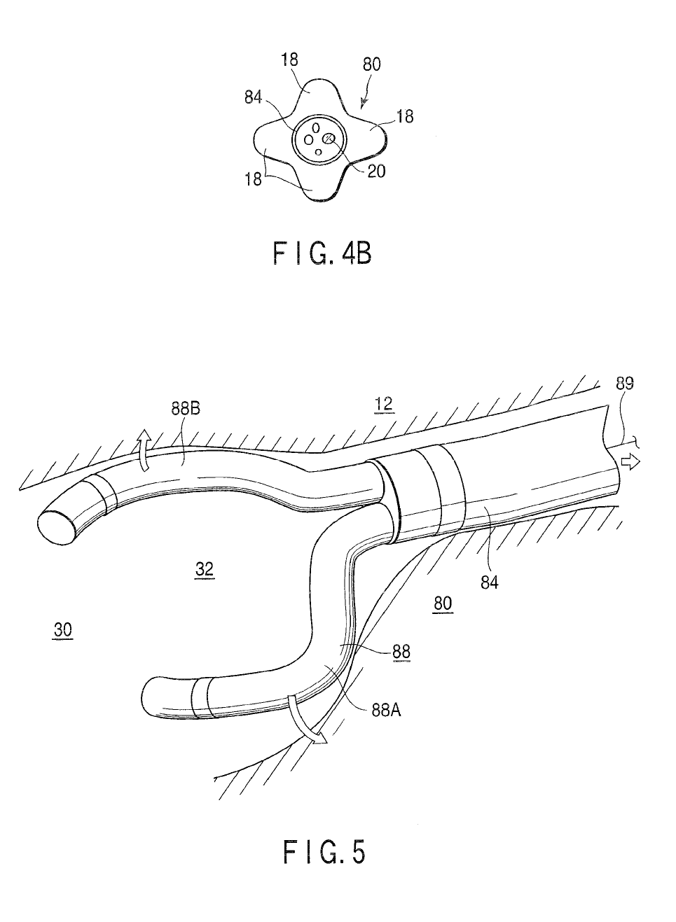

As shown in FIGS. 4A and 4B, a body insertion instrument 10 according to a second embodiment of the present invention has an inserting portion 84 inserted into a body near a target organ 12 such as a heart and balloons 18 disposed to the inserting portion 84.

A small camera 20 is assembled to an extreme end of the inserting portion 84. A configuration for inserting the inserting portion 84 into the body is the same as that for inserting the contact portion 14 and the contact portion support portion 16 of the first embodiment into the body.

Further, a plurality of sets each having four balloons 18 are disposed at intervals in a direction orthogonal to an axial direction of the inserting portion 84 so that the balloons 18 are inflated in four directions orthogonal to each other. A configuration for expanding and contracting the balloons 18 is also the same as that of the first embodiment.

In the body insertion instrument 10 according to the present embodiment having the configuration, when the balloons 18 are inflated, they are expanded in the four directions at respective positions and push the target organ 12 and an adjacent organ 80 and/or a body cavity wall in a direction where they are away from the inserting portion 84. As a result, insertion spaces 86, into which a manipulator 52 is inserted, can be secured between adjacent balloons 18 expanded in the orthogonal directions. Then, since the sets of the balloons 18 are disposed at the intervals and the balloons 18 are expanded as described above, the insertion spaces 86 can be secured as well as operation spaces 32 can be secured between the positions where the balloons 18 are attached.

Note that although each of the four balloons 18 is disposed so that they expand in the four directions at one position, it is needless to say that the expanding directions are not limited to the four directions. Further, the respective balloons may be configured as a single balloon that expands in a plurality of directions in place of that they are configured as independent balloons.

### Third Embodiment

As shown in FIG. 5, a body insertion instrument 10 according to a third embodiment of the present invention has a mechanically openable/closable opening/closing unit 88 attached to an extreme end of an inserting portion 84 inserted into a body near a target organ 12 such as a heat.

A configuration for inserting the inserting portion 84 into the body is the same as that for inserting the contact portion 14 and the contact portion support portion 16 of the first embodiment into the body.

Further, the opening/closing unit 88 includes two curving pipes 88A and 88B connected to a slender cable 89 such as a wire which passes through inside the inserting portion 84. The curving pipes 88A and 88B can be set to a curved mode and a non-curved mode by adjusting tension of the cable 89. Note that the two curving pipes 88A and 88B are attached to the inserting portion 84 so that they are curved in an opposite direction from each other.

In the body insertion instrument 10 according to the embodiment configured as described above, it is inserted into the body in the non-curved mode of the curving pipes 88A and 88B, i.e., in a state that the opening/closing unit 88 is closed. When the cable 89 is pulled in a desired insertion state, the curving pipes 88A and 88B are curved in an opposite direction from each other as shown by arrows in FIG. 5. In the state that the opening/closing unit 88 is opened as described above, when the target organ 12 and an adjacent organ 80 and/or a body cavity wall are pushed by the curving pipes 88A and 88B in a direction where they are away from the inserting portion 84, an operation space 32 continuous to a chest cavity 30 can be secured.

Note that it is needless to say that suction holes 24 may be formed to the curving pipes 88A and 88B as described in the first embodiment to thereby draw the target organ 12 and/or the adjacent organ 80 or the body cavity wall and stabilize the motion thereof. That is, the curving pipes 88A and 88B can be also used as a stabilizer.

Further, in FIG. 5, the cable 89 is composed of a single cable, and the curving pipes 88A and 88B are curved at the same time by pulling the cable 89. However, independent cables may be disposed to the curving pipes 88A, 88B so that the curving pipes 88A and 88B can be moved independently of each other. Further, a cable may be provided in addition to the cable for deforming the curving pipes 88A and 88B to a curved shape so that the curving pipes 88A and 88B can be returned to a state before they are curved (for example, to a straight state).

### [First Modification]

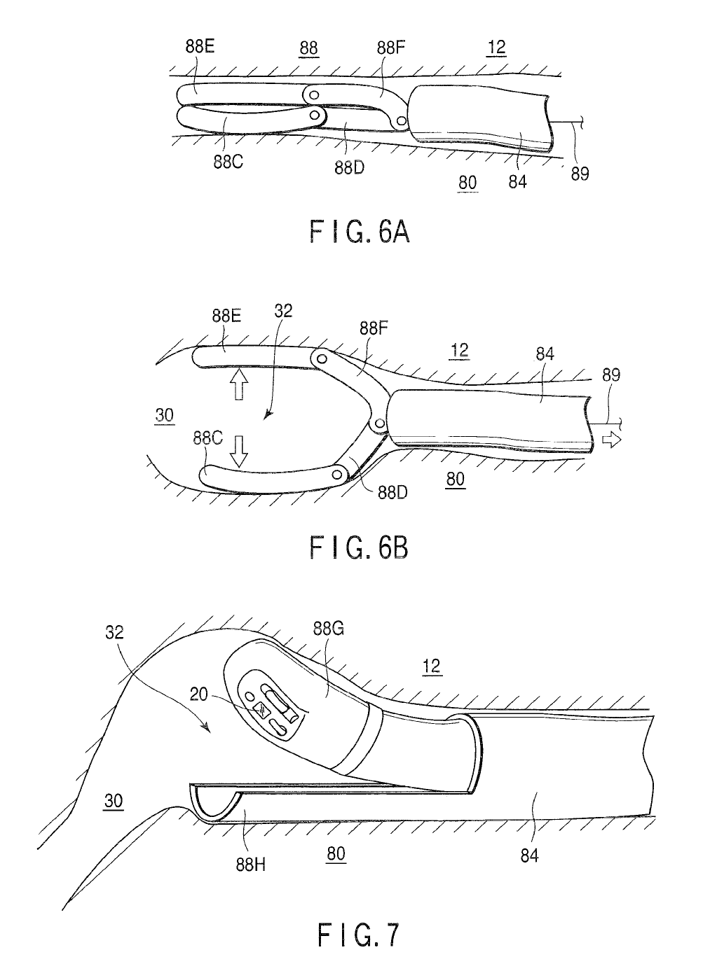

As shown in FIGS. 6A and 6B, a first modification of the present embodiment uses four movable spatulas 88C, 88D, 88E, and 88F to an opening/closing unit 88 in place of the two curving pipes 88A and 88B.

Even if the opening/closing unit 88 uses the movable spatulas 88C, 88D, 88E, and 88F, an operation space 32 can also secured likewise.

### [Second Modification]

As shown in FIG. 7, in a second modification of the embodiment, an opening/closing unit 88 includes one curved pipe 88G and a fixed portion 88H. The fix portion 88H has such a degree of rigidity that it does not obstruct its insertion. Further, a small camera 20 is assembled to the curving pipe 88G so that it is protected by the fixed portion 88H when the curving pipe 88G is not curved and is exposed when it is curved.

An operation space 32 can be secured likewise even by the configuration. Further, the operation space 32 can be also observed by the small camera 20.

Note that it is needless to say that suction holes 24 as described in the first embodiment may be formed to the curving pipe 88G and/or the fixed portion 88H to draw a target organ 12 and/or an adjacent organ 80 or a body cavity wall and to stabilize the motion thereof.

Note that the second modification is embodied in such a configuration, for example, that a side-view type endoscope is used as the curving pipe 88G, an inserting portion 84 is configured as an overtube for covering the endoscope, and the fixed portion 88H is configured by removing a part of an extreme end of the overtube so that the endoscope can be deformed.

### [Third Modification]

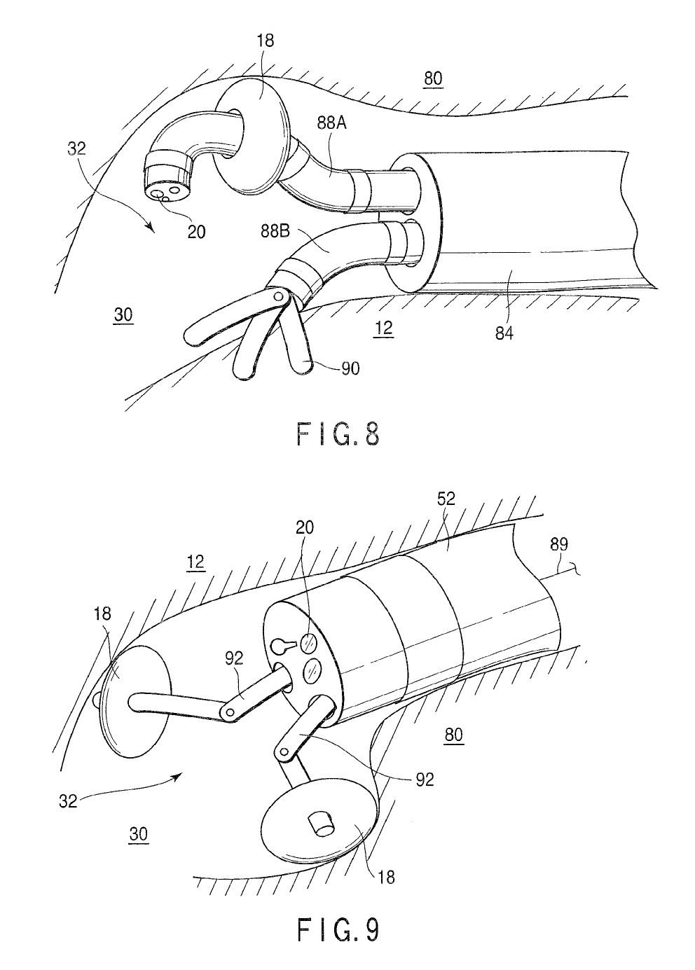

As shown in FIG. 8, in a third modification of the present embodiment, a balloon 18 is disposed to one of two curving pipes 88A, 88B that constitute an opening/closing unit 88, i.e., to the curving pipe 88A, and a flat plate 90, which can be opened and closed in a fan shape as disclosed in Jpn. Pat. Appln. KOKAI Publication No. 06-154152, is disposed to the other curving pipe 88B.

An operation space 32 can be also secured likewise even by the configuration.

Further, motion of the target organ 12 can be stabilized by pushing it by opening the flat plate 90, which is disposed to the curving pipe 88A on the side where it is in contact with the target organ 12, in the fan shape.

Further, it is also possible to observe the operation space 32 as shown in FIG. 8 by arranging the curving pipe 88B as a two-stage curving type pipe and assembling a small camera 20 to an extreme end thereof.

Note that when the third modification is embodied, a manipulator 52 can be used as, for example, each of the curving pipes 88A and 88B, and an inserting portion 84 can be configured as an overtube for covering the manipulators 52.

### Fourth Embodiment

As shown in FIG. 9, a body insertion instrument 10 according to a fourth embodiment of the present invention is provided as a manipulator 52 with a small camera 20 used in an endoscopic operation and has two forceps 92, which can be put into and taken out from an extreme end of the manipulator 52, and balloons 18 disposed to the forceps 92.

The two forceps 92 have a curving function, and a curved state thereof can be adjusted by adjusting tension of a slender cable 89 such as a wire passing through inside the manipulator 52. Note that the two forceps 92 are attached so that they are curved in an opposite direction from each other.

Further, the balloon 18 can be inflated by supplying air thereinto from a supply air pressure transmission path (not shown) disposed in the forceps 92 and the manipulator 52.

In the body insertion instrument 10 according to the present embodiment configured as described above, the forceps 92 and the balloons 18 are inserted into a body in a state that they are accommodated in the manipulator 52. The forceps 92 are extended from the manipulator 52 in a desired inserted state, and the two forceps 92 are curved in the opposite direction from each other by pulling the cable 89. At the time, the curved forceps 92 can be prevented from damaging the body by inflating the balloons 18 at the same time.

An operation space 32 continuous to a chest cavity 30 can be secured by pushing a target organ 12 and an adjacent organ 80 and/or a body cavity wall in a direction where they are separated from an inserting portion 84 by the balloons 18 by curving the forceps 92 up to a desired position as described above.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A body insertion instrument comprising:
an inserting portion adapted to be inserted into a body near a target organ as a target of surgery;
a space securing unit, disposed on the inserting portion, configured to secure a space, in which the target organ is operated on, the space comprising one of between the target organ and an organ different from the target organ and between the target organ and one of an abdominal wall and a chest wall;
a drive unit configured to drive the space securing unit to create a force between the target organ and the organ different from the target organ and between the target organ and at least one of the abdominal wall and the chest wall; and
wherein the space securing unit is disposed on a first side, the first side being where the inserting portion is in contact with the target organ, and on a second side opposite to the first side.

2. The body insertion instrument according to claim 1, wherein the space securing unit is selectively set to a first mode, the first mode at a time before the body insertion instrument is inserted into the body and to a second mode, the second mode at a time after the body insertion instrument is inserted into the body and has, the second mode having a volume larger than that in the first mode.

3. The body insertion instrument according to claim 1, wherein the drive unit creates areas of force between the target organ and the organ different from the target organ and between the target organ and the one of the abdominal wall and the chest wall.

4. The body insertion instrument according to claim 1, wherein the drive unit is adapted to restrict organs in a body cavity, on which force of the space securing unit acts, over a partial area of the organs.

5. The body insertion instrument according to claim 1, wherein the space securing unit comprises a curvable member having at least two curvature functions.

6. The body insertion instrument according to claim 1, wherein the space securing unit includes at least one balloon configured to be inflated in the body.

7. The body insertion instrument according to claim 1, wherein the space securing unit comprises a fixed portion and a curvable member having a curvature function, the fixed portion securing the curvable member in a state where the curvable member is not curved.

8. The body insertion instrument according to claim 1, wherein the drive unit is electric-powered.
